(19) United States
(12) Patent Application Publication
(10) Pub. No.: US 2019/0117431 A1
(43) Pub. Date: Apr. 25, 2019

(54) SPLIT TYPE EXTERNAL FIXATION DEVICE OF CERVICAL AND THORACIC VERTEBRAE

(71) Applicant: Xieping ZHU, Shanghai (CN)

(72) Inventor: Xieping ZHU, Shanghai (CN)

(21) Appl. No.: 15/788,841

(22) Filed: Oct. 20, 2017

(51) Int. Cl. A61F 5/02

(52) U.S. Cl. CPC A61F 5/02

(57) ABSTRACT

A split-type external device for fixation of cervical and thoracic vertebrae, the device including: a first part adapted to cover a neck and a chest of a patient, a second part adapted to cover a head of the patient, and a third part adapted to cover an abdomen and waist of the patient. The first part includes a first left part and a first right part. The first left part and the first right part are symmetrically disposed along a front middle seam and a back middle seam which are corresponding to axial lines on the front and back of the patient; the first left part includes a left neck part, a left upper chest part, a left front part, a left back part, and a left upper back part which are integrated with one another to yield a first left cavity adapted to accommodating filler slurry.

### BACKGROUND OF THE INVENTION

### Field of the Invention

The invention relates to a split-type external device for fixation of cervical and thoracic vertebrae.

### Description of the Related Art

Conventionally, when the patients suffer from acute or chronic damage of cervical and thoracic vertebrae as well as soft tissue damage around the cervical and thoracic vertebrae, a fixation device is utilized for temporary fixation and protection of the fractured bones in order to maintain the normal physiological curvature and avoid further damage.

Typical external fixation mostly adopts tubular plasters and bandages, high polymer bandages, or splints. The fixation by splints often results in malunion or nonunion of the fractured surfaces, leading to functional defects in the fractured bones. Fixation using tubular plaster or high polymer bandages is disadvantageous in the following aspects: complex operation, poor flexibility and breathability of the materials, and loose fitting between the fixation device and the fractured bones. In addition, the X-rays cannot penetrate the plaster or bandage, increasing the difficulty for physicians to monitor bone healing. Furthermore, the tubular plasters tend to cause joint adhesion and ankylosis.

### SUMMARY OF THE INVENTION

To achieve the above objective, in accordance with one embodiment of the invention, there is provided a split-type external device for fixation of cervical and thoracic vertebrae which adapts its shape to the shape of the fractured bones of a patient, is convenient to use, and provides good fixation effect.

To achieve the above objective, in accordance with one embodiment of the invention, there is provided a split-type external device for fixation of cervical and thoracic vertebrae, the device comprising: a first part adapted to cover a neck and a chest of a patient, a second part adapted to cover a head of the patient, and a third part adapted to cover an abdomen and waist of the patient. The first part comprises a first left part and a first right part which are connected to one another to embrace the neck and the chest of the patient.

The first left part and the first right part are symmetrically disposed along a front middle seam and a back middle seam which are corresponding to axial lines on the front and back of the patient;

the first left part comprises a left neck part, a left upper chest part, a left front part, a left back part, and a left upper back part which are integrated with one another to yield a first left cavity adapted to accommodating filler slurry; the second part comprises a second left part and a second right part; the second left part and the second right part are connected to a left winding belt and a right winding belt, respectively; the left neck part extends upwards and outwards to connect to the second left part and the left winding belt;

the first right part comprises a right neck part, a right upper chest part, a right front part, a right back part, and a right upper back part which are integrated with one another to yield a first right cavity adapted to accommodating filler slurry; the right neck part extends upwards and outwards to connect to the second right part and the right winding belt;

a right edge of the left upper chest part of the first left part and a left edge of the right upper chest part of the first right part are connected via front central seam connecting pieces;

a right edge of the left upper back part of the first left part and a left edge of the right upper back part of the first right part are connected via back central seam connecting pieces;

lower ends of the first left part and the first right part are provided with connecting parts; first connecting parts are disposed on a front side of the first left part and the first right part corresponding to the abdomen of the patient, second connecting parts are disposed on a back side of the first left part and the first right part corresponding to the waist of the patient; the first connecting parts are connected to third connecting parts disposed on an upper edge of a front side of the third part, and the second connecting parts are connected to fourth connecting parts disposed on an upper edge of a back side of the third part;

the left winding belt and the right winding belt each are provided with a hook and loop;

the first left part comprises two layers of knitting cloth and a left opening communicating with the first left cavity;

the first right part comprises two layers of knitting cloth and a right opening communicating with the first right cavity.

The polyurethane slurry is filled to the first left cavity of the first left part via a left opening communicating with the first left cavity, and filled to the first right cavity of the first right part via a right opening communicating with the first right cavity. Thereafter, the polyurethane slurry is allowed to solidify while holding the softening state, and then the first left part and the first right part are sewed via the front and back central seam connecting pieces.

The third part comprises a front subpart corresponding to an abdomen of the patient and a back subpart corresponding to a waist of the patient; two sides of the front subpart are provided with elastic belts which are provided with first fasteners; two sides of the back subpart are provided with second fasteners; and the first and second fasteners are hook and loops.

The front subpart and the back subpart each comprise two layers of knitting cloth. The lower ends of the front subpart and the back subpart are provided with hook and loops.

The polyurethane slurry is filled to the front cavity of the front subpart via an opening communicating with the front cavity, and filled to the back cavity of the back subpart via an opening communicating with the back cavity. Thereafter, the polyurethane slurry is allowed to solidify while holding the softening state, and then the front subpart and the back subpart tightened via the connecting pieces to wrap the lower half of the chest and the waist of the patient. The front subpart and the back subpart can adapt its shape to the shape of the fractured bones of the patient.

Two sides of the front subpart and the back subpart extend to a position corresponding to an ilium part of the patient, a bottom end of the front subpart extends to a position corresponding to a groin of the patient, and a bottom end of the back subpart extends to a position corresponding to a caudal vertebra of the patient.

The split-type external device for fixation of cervical and thoracic vertebrae of the invention can be respectively used as an external device for fixation of neck parts or an external device for fixation of thoracic and lumbar vertebrae or be used as an external device for fixation of cervical and thoracic vertebrae. Therefore, the device features a wide application scope, a good fixation effect, is convenient for use, can rapidly adapt its shape to the fractured bones of patients, fit curves of human bodies closely so as to realize reliable fixation and protection of fractured bones of cervical and thoracic vertebrae of patients or preoperative and postoperative parts of patients and maintain the normal physiological curvature of cervical and thoracic vertebrae. The invention can be used for fixation in treatment of severe cervical vertebrae damage, for movement protection during recovery from cervical vertebrae damage, for patients who have higher requirements on cervical vertebrae, for patients who need to restrict rotation of the upper segments of cervical vertebrae and for fixation of fractured upper thoracic vertebrae and luxated and subluxated cervical vertebrae after reduction.

### BRIEF DESCRIPTION OF THE DRAWINGS

FIG. 1 is a front view of a split-type external device for fixation of cervical and thoracic vertebrae of the disclosure;

FIG. 2 is a rear view of a split-type external device for fixation of cervical and thoracic vertebrae of the disclosure; and

FIG. 3 is a side view of a split-type external device for fixation of cervical and thoracic vertebrae of the disclosure.

### DETAILED DESCRIPTION OF THE EMBODIMENTS

The invention is described in detail below with reference to the accompanying drawings and embodiments.

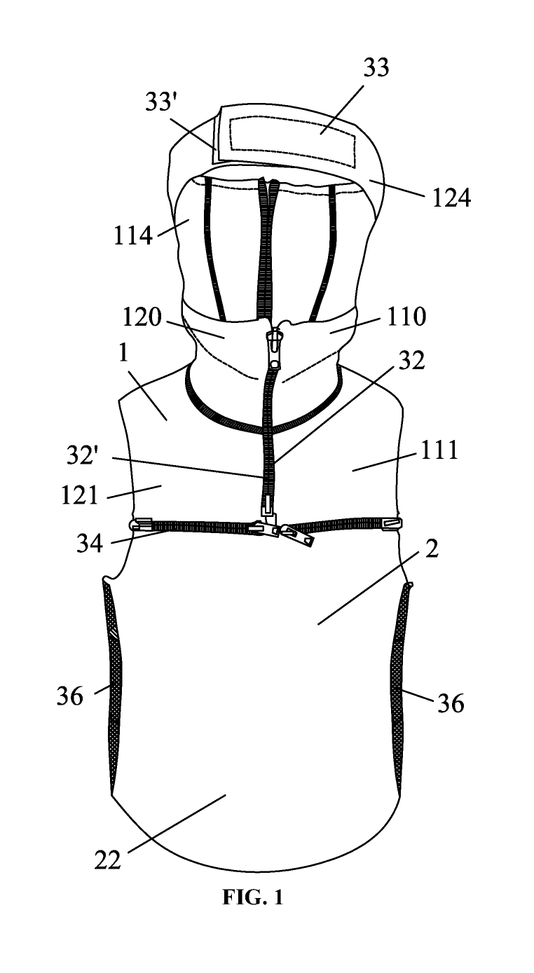

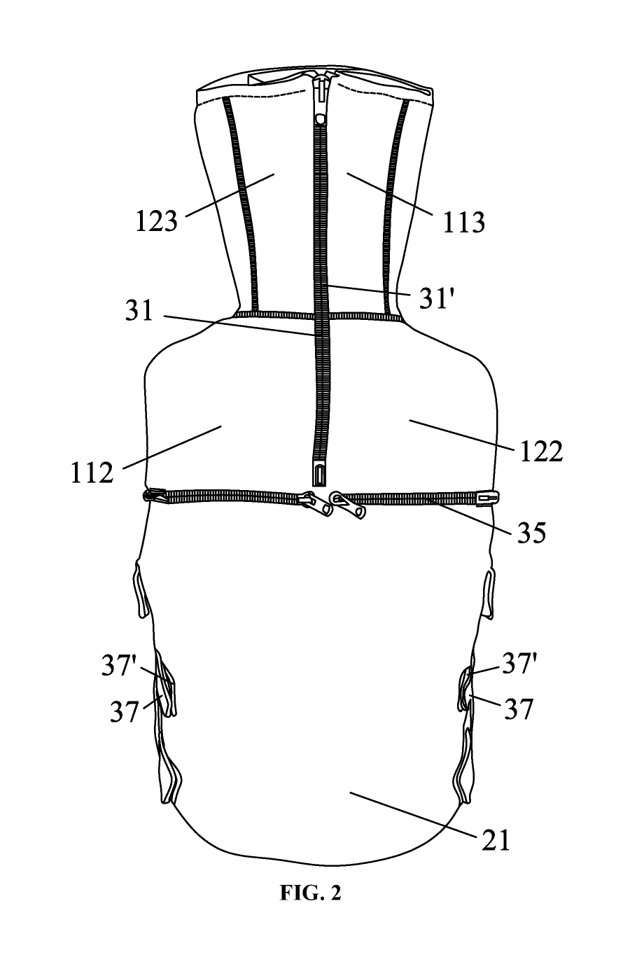

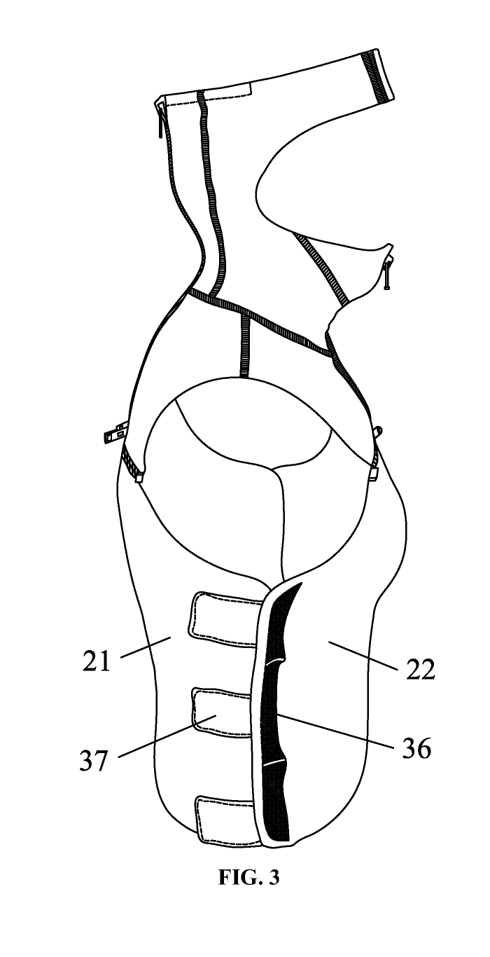

Please refer to FIGS. 1, 2 and 3, a split-type external device for fixation of cervical and thoracic vertebrae is a fixation structure which adapts to change its physical appearance in conformity to the shape of the fractured bones of a patient. The device comprises a first part 1 adapted to cover a neck and a chest of a patient, a second part 2 adapted to cover a head of the patient, and a third part adapted to cover an abdomen and waist of the patient.

The first part is a fixation unit capable of change its physical shape in conformity to the shape of the fractured bones of a patient, and comprises a first left part 110 and a first right part 120 which are connected with one another to embrace the neck and the chest of the patient.

The first left part and the first right part are symmetrically disposed along a front middle seam and a back middle seam which are corresponding to axial lines on the front and back of the patient. The first left part and the first right part can be unfolded to form a flat structure either prior to or after being filled with filler slurry.

The first left part 110 comprises a left neck part, a left upper chest part 111, a left front part, a left back part, and a left upper back part which are integrated with one another to yield a first left cavity adapted to accommodating filler slurry; the second part comprises a second left part 113 and a second right part; the second left part and the second right part are connected to a left winding belt and a right winding belt 114, respectively; the left neck part extends upwards and outwards to connect to the second left part 113 and the left winding belt 114;

the first right part 120 comprises a right neck part, a right upper chest part 121, a right front part, a right back part, and a right upper back part which are integrated with one another to yield a first right cavity adapted to accommodating filler slurry; the right neck part extends upwards and outwards to connect to the second right part 123 and the right winding belt 124;

a right edge of the left upper chest part 111 of the first left part and a left edge of the right upper chest part 121 of the first right part are connected via front central seam connecting pieces 32, 32′;

a right edge of the left upper back part 112 of the first left part and a left edge of the right upper back part 122 of the first right part are connected via back central seam connecting pieces 31, 31′; both the back central seam connecting pieces 31, 31′ and the front central seam connecting pieces 32, 32′ are both zippers.

the lower ends of the first left part and the first right part are provided with connecting parts; first connecting parts 34 are disposed on a front side of the first left part and the first right part corresponding to the abdomen of the patient, second connecting pieces 35 are disposed on a back side of the first left part and the first right part corresponding to the waist of the patient; the first connecting parts are connected to third connecting parts disposed on an upper edge of a front side of the third part, and the second connecting parts are connected to fourth connecting parts disposed on an upper edge of a back side of the third part;

the left winding belt 114 and the right winding belt 124 each are provided with a hook and loop 33, 33′;

the first left part 110 comprises two layers of knitting cloth and a left opening communicating with the first left cavity;

the first right part 120 comprises two layers of knitting cloth and a right opening communicating with the first right cavity.

The polyurethane slurry is filled to the first left cavity of the first left part via a left opening communicating with the first left cavity, and filled to the first right cavity of the first right part via a right opening communicating with the first right cavity. Thereafter, the polyurethane slurry is allowed to solidify while holding the softening state, and then the first left part and the first right part are sewed via the front central seam connecting pieces 32, 32′ and back central seam connecting pieces 33, 33′.

The third part comprises a front subpart 22 corresponding to an abdomen of the patient and a back subpart 21 corresponding to a waist of the patient; two sides of the front subpart are provided with elastic belts 36 which are provided with first fasteners 37; two sides of the back subpart are provided with second fasteners 37′; and the first and second fasteners 37, 37′ are hook and loops.

The front subpart 22 and the back subpart 21 each comprise two layers of knitting cloth. The lower ends of the front subpart and the back subpart are provided with hook and loops.

The polyurethane slurry is filled to the front cavity of the front subpart 22 via an opening communicating with the front cavity, and filled to the back cavity of the back subpart 21 via an opening communicating with the back cavity. Thereafter, the polyurethane slurry is allowed to solidify while holding the softening state, and then the front subpart and the back subpart tightened via the connecting pieces to wrap the lower half of the chest and the waist of the patient. The front subpart and the back subpart can adapt its shape to the shape of the fractured bones of the patient.

The application method of a split-type external device for fixation of cervical and thoracic vertebrae of the invention is described as follows.

When the external device is used for fixation of fractured neck parts, firstly, specially prepared polyurethane A slurry and polyurethane B slurry are mixed to produce polyurethane slurry (disclosed in the U.S. Pat. No. 9,617,370); then the first left part 110 and the first right part 120 are filled with the polyurethane slurry; the polyurethane slurry is rolled and evened on an operating platform; after the polyurethane slurry enters a solid-phase stage, the first left part 110 and the first right part 120 are connected via the back central seam connecting pieces 31 and 31′ to form a chest cushion, back cushion, jaw support, and occiput support; the first left part 110 and the first right part 120 wrap the neck part and the upper chest part of the human body and are tightened through the front central seam connecting piece 32 and 32′; then the left winding belt 114 and the right winding belt 124 wrap the head of the human body; the hook and loop fastener 33′ at the end of the left winding belt 114 and the hook and loop fastener 33 at the right winding belt 124 are pressed together to straighten the head.

When the external device is used for fixation of thoracic and lumbar vertebrae, specially prepared polyurethane A slurry and polyurethane B slurry are mixed to produce polyurethane slurry; then the front subpart 22 and the back subpart 21 are filled with the polyurethane slurry; the polyurethane slurry is rolled and evened on an operating platform; after the polyurethane slurry enters a solid-phase stage, the fixation device in a proper size is made on site to wrap the lower chest part and the waist of a human body.

When the external device is used for fixation of cervical and thoracic vertebrae, an external device for fixation of neck parts and an external device for fixation of thoracic and lumbar vertebrae are made first; then the two external devices are combined through connecting pieces.

The split-type external device for fixation of cervical and thoracic vertebrae of the invention can be respectively used as an external device for fixation of neck parts or an external device for fixation of thoracic and lumbar vertebrae or be used as an external device for fixation of cervical and thoracic vertebrae. Therefore, the device features a wide application scope, a good fixation effect, is convenient for use, can rapidly adapt its shape to the fractured bones of patients, fit curves of human bodies closely so as to realize reliable fixation and protection of fractured bones of cervical and thoracic vertebrae of patients or preoperative and postoperative parts of patients and maintain the normal physiological curvature of cervical and thoracic vertebrae. The invention can be used for fixation in treatment of severe cervical vertebrae damage, for movement protection during recovery from cervical vertebrae damage, for patients who have higher requirements on cervical vertebrae, for patients who need to restrict rotation of the upper segments of cervical vertebrae and for fixation of fractured upper thoracic vertebrae and luxated and subluxated cervical vertebrae after reduction.

While particular embodiments of the invention have been shown and described, it will be obvious to those skilled in the art that changes and modifications may be made without departing from the invention in its broader aspects, and therefore, the aim in the appended claims is to cover all such changes and modifications as fall within the true spirit and scope of the invention.

The invention claimed is:

1. An external fixation device for fixation of cervical and thoracic vertebrae, the device comprising: a first part adapted to cover a neck and a chest of a patient, a second part adapted to cover a head of the patient, and a third part adapted to cover an abdomen and waist of the patient;
wherein:
the first part comprises a first left part and a first right part which are connected to one another to embrace the neck and the chest of the patient;
the first left part and the first right part are symmetrically disposed along a front middle seam and a back middle seam which are corresponding to axial lines on the front and back of the patient;
the first left part comprises a left neck part, a left upper chest part, a left front part, a left back part, and a left upper back part which are integrated with one another to yield a first left cavity adapted to accommodating filler slurry; the second part comprises a second left part and a second right part; the second left part and the second right part are connected to a left winding belt and a right winding belt, respectively; the left neck part extends upwards and outwards to connect to the second left part and the left winding belt;
the first right part comprises a right neck part, a right upper chest part, a right front part, a right back part, and a right upper back part which are integrated with one another to yield a first right cavity adapted to accommodating filler slurry; the right neck part extends upwards and outwards to connect to the second right part and the right winding belt;
a right edge of the left upper chest part of the first left part and a left edge of the right upper chest part of the first right part are connected via front central seam connecting pieces;
a right edge of the left upper back part of the first left part and a left edge of the right upper back part of the first right part are connected via back central seam connecting pieces;
lower ends of the first left part and the first right part are provided with connecting parts; first connecting parts are disposed on a front side of the first left part and the first right part corresponding to the abdomen of the patient, second connecting parts are disposed on a back side of the first left part and the first right part corresponding to the waist of the patient; the first connecting parts are connected to third connecting parts disposed on an upper edge of a front side of the third part, and the second connecting parts are connected to fourth connecting parts disposed on an upper edge of a back side of the third part;
the left winding belt and the right winding belt each are provided with a hook and loop;
the first left part comprises two layers of knitting cloth and a left opening communicating with the first left cavity;
the first right part comprises two layers of knitting cloth and a right opening communicating with the first right cavity;
the third part comprises a front subpart corresponding to an abdomen of the patient and a back subpart corresponding to a waist of the patient;
two sides of the front subpart are provided with elastic belts which are provided with first fasteners; two sides of the back subpart are provided with second fasteners; and the first and second fasteners are hook and loops.

2. The device of claim 1, wherein two sides of the front subpart and the back subpart extend to a position corresponding to an ilium part of the patient, a bottom end of the front subpart extends to a position corresponding to a groin of the patient, and a bottom end of the back subpart extends to a position corresponding to a caudal vertebra of the patient.

3. The device of claim 1, wherein the back central seam connecting pieces and the front central seam connecting pieces are both zippers.

4. The device of claim 1, wherein the front subpart and the back subpart each comprise two layers of knitting cloth.
